(19) United States
(12) Patent Application Publication
(10) Pub. No.: US 2008/0045968 A1
(43) Pub. Date: Feb. 21, 2008

(54) Instruments and Methods for Spinal Surgery

(75) Inventors: Kidong Yu, Memphis, TN (US); Danny Braddock, Germantown, TN (US)

Correspondence Address: HAYNES AND BOONE, LLP, 901 Main Street, Suite 3100, Dallas, TX, 75202, US

(73) Assignee: WARSAW ORTHOPEDIC, INC., Warsaw, IN (US)

(21) Appl. No.: 11/465,541

(22) Filed: Aug. 18, 2006

(51) Int. Cl. A61F 2/00

(52) U.S. Cl. 606/99

(57) ABSTRACT

A surgical instrument for use in spinal surgery is provided. The surgical instrument may have particular application in posterior total disc arthroplasty. The surgical instrument is adapted to be minimally invasive in some embodiments. The surgical instrument is adapted to maintain the relative positions of multiple components of a prosthesis during insertion. The surgical instrument can be anchored into position by a stability pin. Further, the surgical instrument can guide a drill or other instrument to a portion vertebra to facilitate the securing of the prosthesis components to the vertebrae.

### TECHNICAL FIELD

Embodiments of the invention relate generally to devices and methods for accomplishing spinal surgery, and more particularly in some embodiments, to devices and methods of stabilizing a prosthesis for use in spinal arthroplasty. Various implementations of the invention are envisioned, including use in total spine arthroplasty for stabilizing multiple components of a prosthesis, inserting a prosthesis, and aligning fixation components for securing the prosthesis.

### BACKGROUND

Often spinal implants are comprised of multiple components. For example, some spinal implants include separate upper and lower components that work together to preserve at least some vertebral motion. For these prostheses to function properly, often the upper and lower components must be substantially aligned. However, fixation of the upper and lower components to the natural bone can lead to misalignment and, therefore, improper functioning of the prosthesis.

Accordingly, there is a need for improved instrumentation and methods that avoid the drawbacks and disadvantages of the known methods, devices, and surgical techniques.

### SUMMARY

In one embodiment, a surgical instrument for inserting an implant is provided.

In a second embodiment, a surgical instrument for inserting an implant between adjacent vertebrae is provided. The surgical instrument comprises a first grasping member having a first channel for slidably receiving a first portion of a guide tube; a second grasping member opposed to the first grasping member, the second grasping member having a second channel opposed to the first channel for slidably receiving a second portion of the guide tube; and an actuator adapted for moving the first and second grasping members between a first position for securely grasping the implant and a second position for releasing the implant.

In a third embodiment, a method of inserting a spinal implant at least partially into the intervertebral space between a first vertebra and a second vertebra is provided. The method comprises: providing a surgical instrument comprising a grasping portion movable between a first position for securely grasping the spinal implant and a second position for releasing the spinal implant and a first channel disposed adjacent the grasping portion, the first channel adapted for slidably receiving a portion of a guide tube; creating a first exposure through a patient's back to access the intervertebral space; grasping the spinal implant with the surgical instrument; inserting the spinal implant at least partially into the invertebral space; sliding the portion of the guide tube into the first channel; and securing the spinal implant to the first vertebra.

Additional and alternative features, advantages, uses, and embodiments are set forth in or will be apparent from the following description, drawings, and claims.

### BRIEF DESCRIPTION OF THE DRAWINGS

FIG. 1 is a perspective view of a surgical instrument according to one embodiment of the present application shown with guide tubes and a drill bit.

FIG. 2 is a perspective view of the surgical instrument of FIG. 1.

FIG. 3 is a top view of the surgical instrument of FIG. 1.

FIG. 4 is a side view of the surgical instrument of FIG. 1.

FIG. 5 is a side view of a portion of the surgical instrument of FIG. 1.

FIG. 6 is a top view of a guide tube according to one embodiment of the present application.

FIG. 7 is an end view of the guide tube of FIG. 6.

FIG. 8 is a side view of the apparatus utilized in a method according to one or more aspects of the present application.

FIG. 9 is a side view of the apparatus utilized in another stage of the method according to one or more aspects of the present application.

FIG. 10 is a side view of the apparatus of FIG. 9 utilized in another stage of the method according to one or more aspects of the present application.

FIG. 11 is a side view of the apparatus of FIGS. 9 and 10 utilized in another stage of the method according to one or more aspects of the present application.

FIG. 12 is a side view of a prosthesis inserted by a method according to one or more aspects of the present application.

### DESCRIPTION

For the purposes of promoting an understanding of the principles of the invention, reference will now be made to the embodiments, or examples, illustrated in the drawings and specific language will be used to describe the embodiments. It will nevertheless be understood that no limitation of the scope of the invention is intended. Any alterations and further modifications of the described embodiments, and any further applications of the principles of the invention as described herein are contemplated as would normally occur to one skilled in the art to which the invention relates.

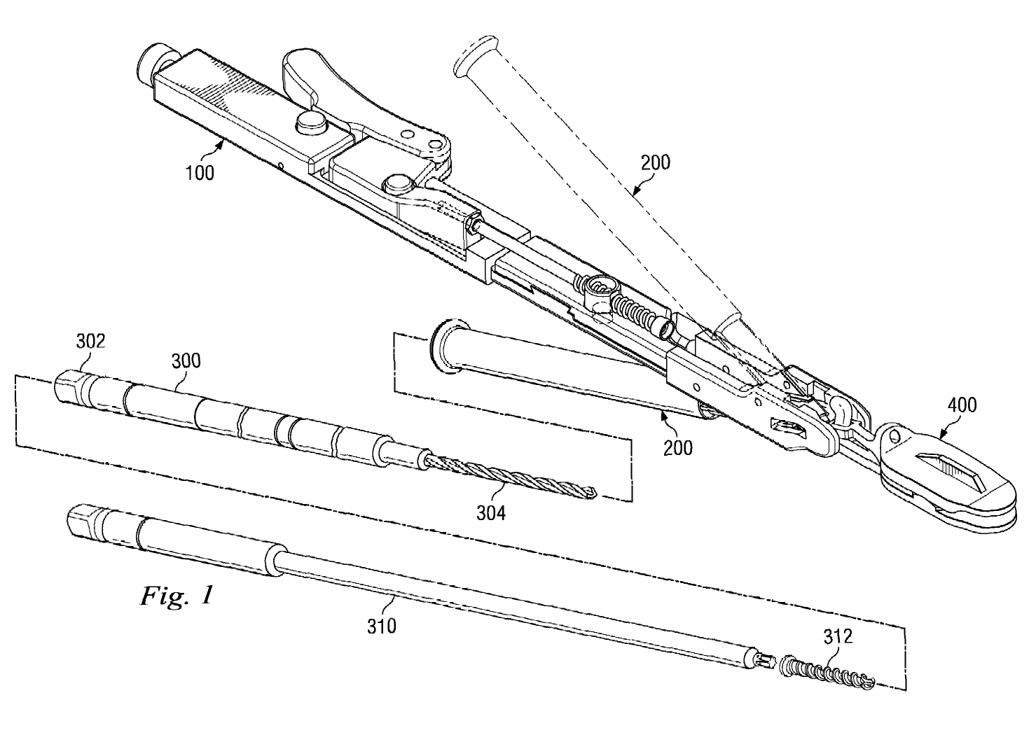

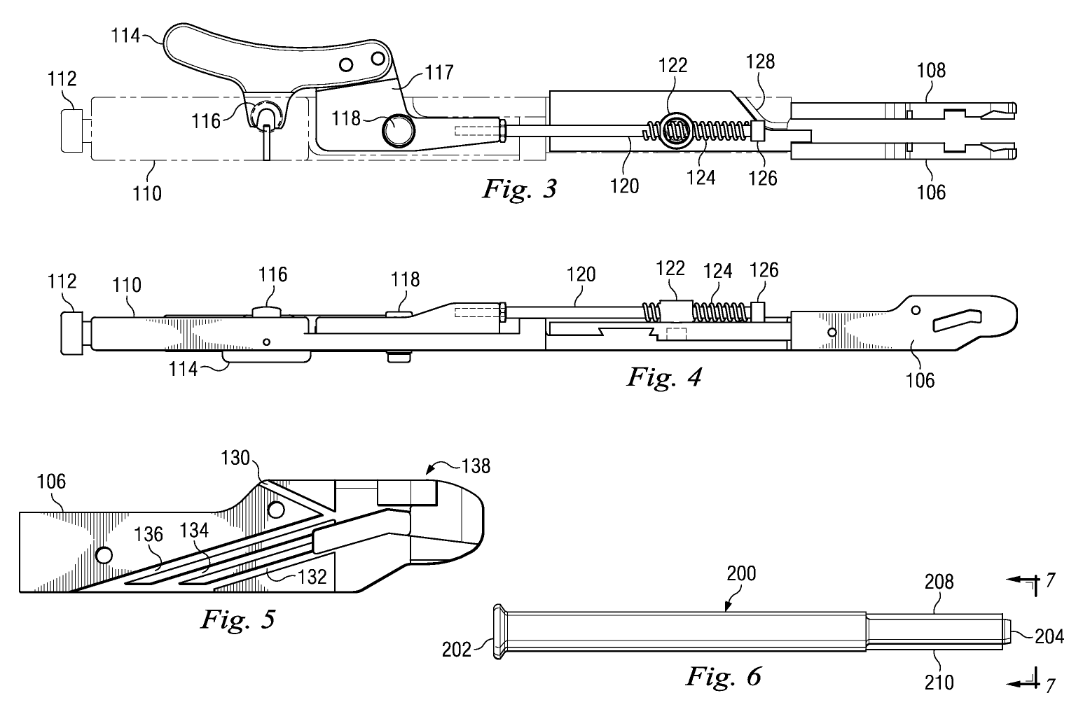

Referring now to FIGS. 1-5, shown therein is an exemplary embodiment of a surgical instrument 100 according to the present invention. Also shown in FIG. 1 in combination with the surgical instrument 100 are a guide tube 200, a drill portion 300, a screw driver 310, and a screw 312. The surgical instrument 100, the guide tube 200, the drill portion 300, the screw driver 310, and the screw 312 are utilized to insert an implant 400. The specific functions of the guide tube 200 and the drill portion 300 in relation to the surgical instrument 100 will be described in greater detail below.

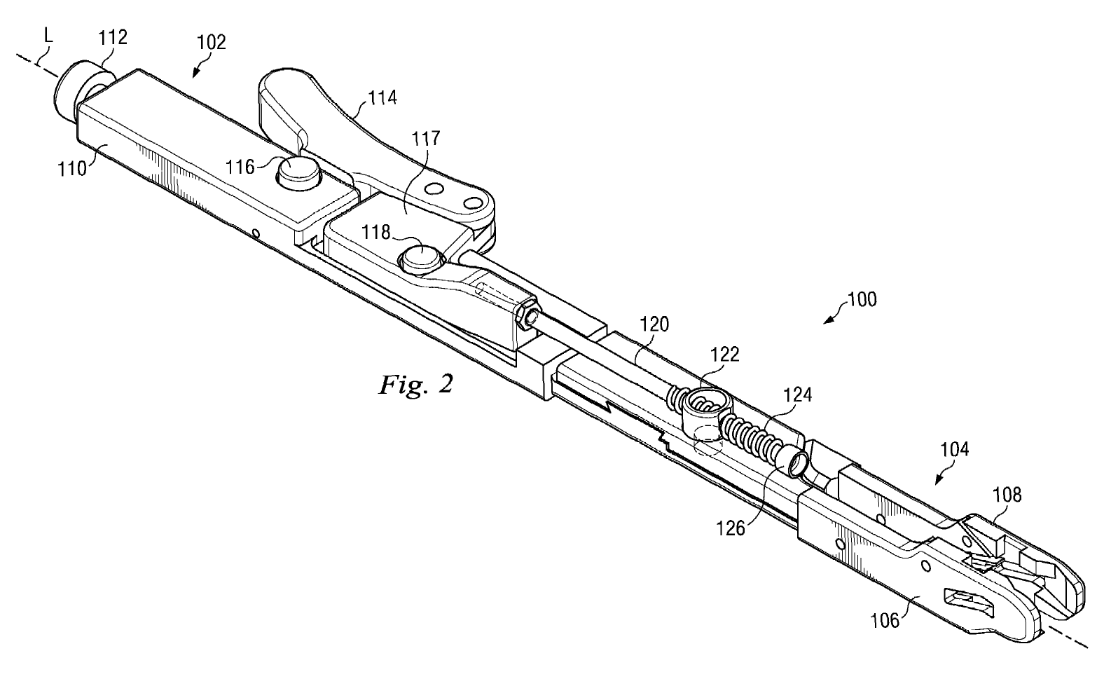

Referring more specifically to FIGS. 2-4, shown therein are various views of the surgical instrument 100. The surgical instrument 100 includes a proximal portion 102, a distal or working portion 104, and a longitudinal axis L extending substantially between the proximal portion and the distal portion. The distal portion 104 comprises two opposed gripping members 106, 108. The gripping members 106, 108 are moveable between a first position for grasping an implant or prosthesis 400 and a second position for releasing the implant or prosthesis. The proximal portion 102 includes a handle 110 and a projection 112. The handle 110 is adapted for grasping by the user or engagement with another instrument. The projection 112 is adapted to receive a force from a mallet, hammer, slap-hammer or similar device that is transferred along the length of the surgical instrument and used to urge the implant 400 into position.

The surgical instrument 100 also includes a mechanism for moving the gripping members 106, 108 between positions for grasping and releasing the prosthesis. In at least one embodiment, the mechanism includes an actuator 114, a body portion 117, and an elongated shaft 120. In the current embodiment, the actuator 114 is positioned adjacent the handle 110 and is adapted for rotational movement about its pivot point 116. The rotational movement of the actuator 114 about pivot point 116 is transferred via body portion 117 and pivot point 118 into substantially linear motion of the elongated shaft 120 substantially along the longitudinal axis L of the surgical instrument. In some embodiments, a rotating member 122 helps facilitate the linear movement of the shaft 120. Also in some embodiments, a spring 124 is positioned along the shaft 120.

The elongated shaft 120 includes an engagement portion 126. The engagement portion 126 is adapted to engage a surface 128. The engagement portion 126 and the surface 128 are oriented such that the engagement between the two causes the gripping members 106, 108 to move between positions for gripping and releasing the prosthesis. In the current embodiment, the surface 128 is angled such that as the elongated shaft 120 extends along the longitudinal axis L towards the distal portion 104 the engagement portion 126 will force the surface 128 outward causing the gripping members 106, 108 to spread apart into a position for releasing the implant 400. In other embodiments, this functionality can be reversed such that as the elongated shaft 120 extends along the longitudinal axis L towards the distal portion 104 the engagement portion 126 forces the surface 128 outward causing distal portion of the gripping members 106, 108 to move towards each other into a position for securing the implant 400 between the gripping members. The actuator 114 and shaft 120 may be replaced by any other mechanism or components capable of moving the gripping members 106, 108 between a grasping position and a releasing position.

In some embodiments, the surgical instrument 100 and the handle 110 are adapted to be held by a single hand of the user and the actuator 114 is adapted for movement by the user's thumb. In one aspect, the actuator 114 includes a locking mechanism (not shown) for selectively securing the actuator in a position. For example, by securing the actuator in a position where the gripping members 106, 108 are engaging the prosthesis the user need not hold the actuator to maintain a secure grip upon the prosthesis. In another aspect, the actuator 114 is biased towards a particular position, such as one corresponding to the grasping or releasing of the prosthesis.

Referring to FIG. 5, shown therein is a side view of the gripping member 106. It should be noted that gripping member 108 is substantially similar to gripping member 106 and will not be described in great detail for that reason. In some embodiments, the gripping member 108 is a mirror image of gripping member 106. It should be noted, however, that gripping member 108 is substantially different from gripping member 106 in some embodiments. The gripping members 106, 108 work together to grasp and release the implant 400 and to position the guide tube 200 appropriately with respect to the implant.

The gripping member 106 includes a superior channel 130, a plurality of inferior channels 132, 134, 136, and implant engagement features 138. The implant engagement features 138 are generally shapes, contours, recesses, projections, textures, or other features that facilitate a secure engagement between the surgical instrument 100 and the implant 400. The implant engagement features 138 may be tailored specifically for a particular implant or adapted to accept a variety of implants. In some embodiments, the implant engagement features 138 help prevent any movement of the components of the implant 400 with respect to other components of the implant or the surgical instrument 100 during insertion. For example, where the implant 400 is a motion preserving implant the implant may have multiple components adapted to move with respect to one another. The implant engagement features 138 of the gripping members 106, 108 serve to secure the components of the implant in a fixed orientation during insertion. This facilitates proper alignment of the implant components all the way through insertion and fixation.

The superior channel 130 of the gripping member 106 is adapted to orient the guide tube 200 such that the guide tube provides access to an inferior portion of the implant 400 (as shown in shadow in FIG. 1). In some embodiments, the superior channel 130 positions the guide tube 200 to facilitate fastening of the implant 400 to an inferior vertebra. Similarly, the inferior channels 132, 134, 136 are adapted to orient the guide tube 200 such that the guide tube provides access to a superior portion of the implant 400 (as shown in FIG. 1). In some embodiments, one of the inferior channels 132, 134, 136 positions the guide tube 200 to facilitate fastening of the implant 400 to a superior vertebra. As the implant 400 is held securely with respect to the gripping members 106, 108 the channels 130, 132, 134, 136 provide precise alignment of the guide tube 200 with features of the implant. For example, in some embodiments the channels 130, 132, 134, 136 provide precise alignment with an engagement portion of the implant, such as opening adapted to receive fixation screw. In some embodiments, the implant 400 itself serves as a stop to limit the travel of the guide tube 200. In other embodiments, the gripping member 106 or other portion of the surgical instrument 100 includes a stop portion to limit the travel of the guide tube 200.

Having a plurality of the inferior channels 132, 134, 136 allows the surgical instrument 100 to be utilized with a variety of implant sizes and types. While in the current embodiment the plurality of inferior channels 132, 134, 136 are substantially parallel, in other embodiments the channels may have different angles providing additional approach angles for the guide tube 200. In other embodiments, the gripping member 106 includes a plurality of superior channels or no superior channels. Further, in other embodiments the gripping member 106 includes none, one, or more inferior channels. Also the channels may be replaced by other features such as notches, protrusions, or other mechanisms adapted to engage with a portion of the guide tube 200 to align the guide tube.

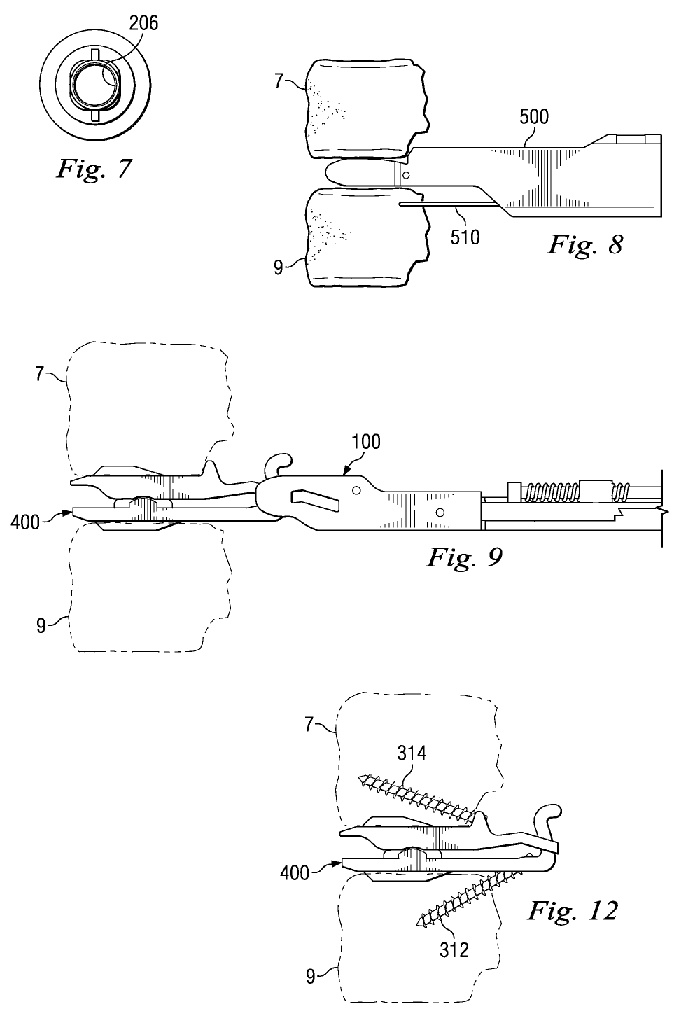

Referring to FIGS. 6 and 7, the guide tube 200 includes a proximal end 202 and a distal end 204. A passage 206 extends substantially from the proximal end 202 to the distal end 204. Adjacent the distal end 204 are flanges 208, 210. The flanges 208, 210 are adapted to slidably mate with the channels of gripping members 106, 108. In other embodiments, the flanges may be replaced by other features in order to facilitate alignment of the guide tube 200 with respect to the surgical instrument 100. In some embodiments the passage 206 of the guide tube 200 provides access and a working space to a portion of the implant 400 and the adjacent vertebra—such as an inferior portion of the implant and the inferior vertebra or the superior portion of the implant and the superior vertebra. This working passage is utilized to prepare the vertebra and to secure the implant 400 to the vertebra. For example, a drill may be inserted through the passage 106 to tap or pre-drill the bone. Then the drill may be removed and a screw inserted through the passage 106 to secure the implant to the bone.

The surgical instrument 100 is made of materials suitable for surgical procedures. For example, in some aspects metals, such as stainless steel and titanium, and polymers, such as polyetheretherketone (PEEK), are used.

Referring now to FIGS. 8-12, shown therein are various stages of a method for inserting an implant according to one aspect of the present disclosure. The surgical instrument 100 described above may be utilized with a wide variety of types and styles of implants. However, the surgical instrument 100 is particularly useful in the insertion of spinal implants having multiple components. For example, in some embodiments the surgical instrument 100 is adapted for inserting the implants and prostheses described in the following patent applications, herein incorporated by reference in their entirety:
U.S. Utility patent application Ser. No. 11/031,602, filed on Jan. 7, 2005 and entitled “Spinal Arthroplasty Device and Method;”
U.S. Utility patent application Ser. No. 11/031,603, filed on Jan. 7, 2005 and entitled “Dual Articulating Spinal Device and Method;”
U.S. Utility patent application Ser. No. 11/031,780, filed on Jan. 7, 2005 and entitled “Split Spinal Device and Method;”
U.S. Utility patent application Ser. No. 11/031,904, filed on Jan. 7, 2005 and entitled “Interconnected Spinal Device and Method;”
U.S. Utility patent application Ser. No. 11/031,700, filed on Jan. 7, 2005 and entitled “Support Structure Device and Method;”
U.S. Utility patent application Ser. No. 11/031,783, filed on Jan. 7, 2005 and entitled “Mobile Bearing Spinal Device and Method;”
U.S. Utility patent application Ser. No. 11/031,781, filed on Jan. 7, 2005 and entitled “Centrally Articulating Spinal Device and Method;”
U.S. Utility patent application Ser. No. 11/031,903, filed on Jan. 7, 2005 and entitled “Posterior Spinal Device and Method;” and
U.S. Utility patent application Ser. No. 11/494,311, 524 filed on Jul. 27, 2006 and entitled “Prosthetic Device for Spinal Joint Reconstruction.”

FIG. 8 shows a preparation step where the vertebral end plates of a superior vertebra 7 and an inferior vertebra 9 are prepared. An instrument 500 and a stability pin 510 may be utilized to prepare the vertebrae 7, 9. The preparation may include removing portions of the vertebrae 7, 9 or contouring the surfaces of the vertebrae to receive the implant or portions of the implant. For example, in some embodiments, preparation of the vertebrae 7, 9 includes creating a channel in at least one of the vertebra to receive a keel of the implant. In at least one embodiment, the preparation of the vertebrae 7, 9 is substantially similar to that described in U.S. Utility Patent Application Attorney Docket No. P23531.00/31132.449 filed on Mar. 30, 2006 and entitled “Instruments and Methods for Preparing an Intervertebral Space” herein, incorporated by reference in its entirety.

FIG. 9 shows the implant 400 being inserted between the vertebrae 7, 9. The implant 400 is being securely held by the surgical instrument 100. As shown, the gripping members 106, 108 of the surgical instrument 100 grasp the implant 400 along the sides of the implant. As previously described, the gripping members 106, 108 include features to prevent movement of the components of the implant 100 during insertion. The implant 400 is secured between the gripping members 106, 108 by positioning the implant 400 between the gripping members while the gripping members are in a releasing position and moving the actuator 114 to cause the gripping members to move to a grasping position to grasp the implant. In some embodiments, the actuator 114 is secured or locked in place by a locking mechanism to securely grasp the implant 400 without the user having to continuously hold the actuator. Further, as described above the gripping members 106, 108 includes additional implant engagement members 138 to prevent movement or slippage of the implant 400.

Also, the surgical instrument 100 includes a projection 112. If necessary, the projection 112 is adapted to receive a force from a mallet, hammer, slap-hammer or similar device that is transferred along the length of the surgical instrument and used to urge the implant 400 into position between the vertebrae 7, 9. In some aspects, once the implant 400 is positioned between the vertebrae 7, 9 the surgical instrument 100 may be released by the user as the force between the implant and the vertebrae will hold the surgical instrument in place. In other embodiments, a stability pin or other device may be used to hold the surgical instrument in place. For a more detailed description of the use of a stability pin to position a surgical instrument, see U.S. Utility Patent Application Attorney Docket No. P23531.00/31132.449 filed on Mar. 30, 2006 and entitled “Instruments and Methods for Preparing an Intervertebral Space,” incorporated by reference in its entirety above.

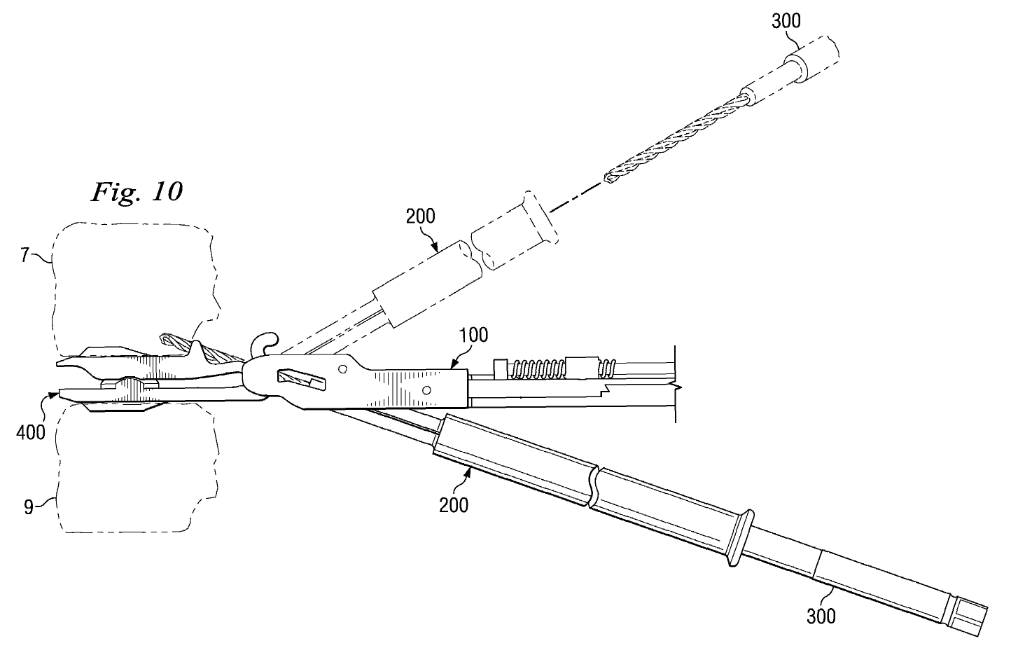

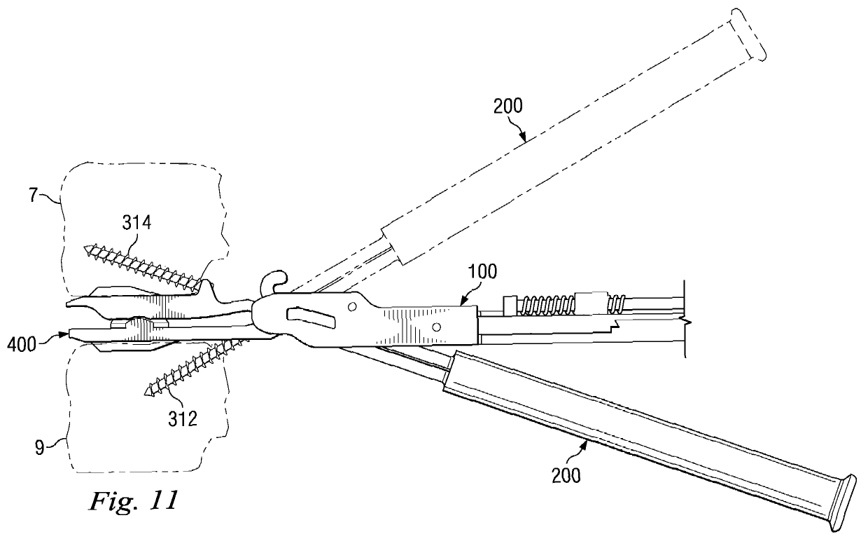

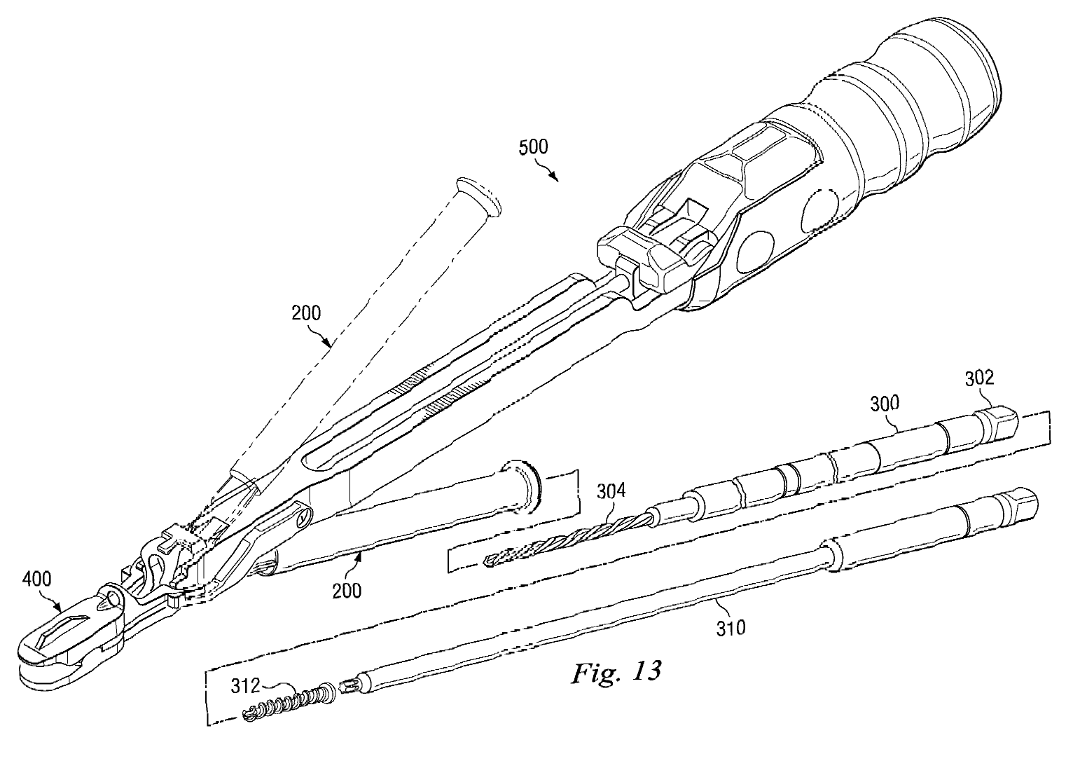

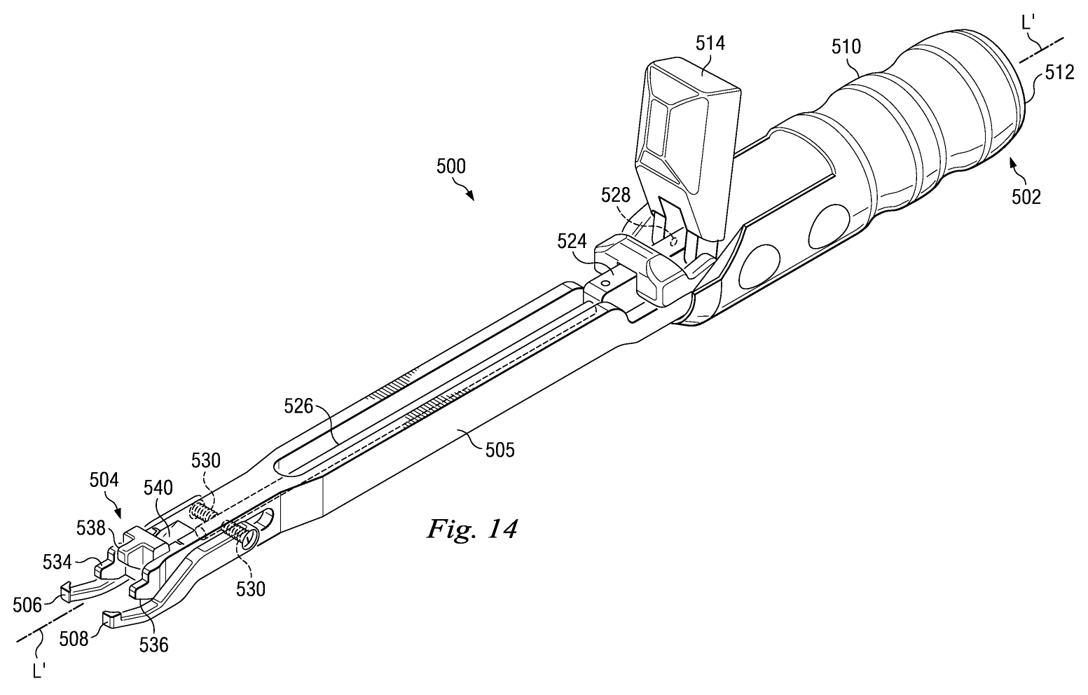

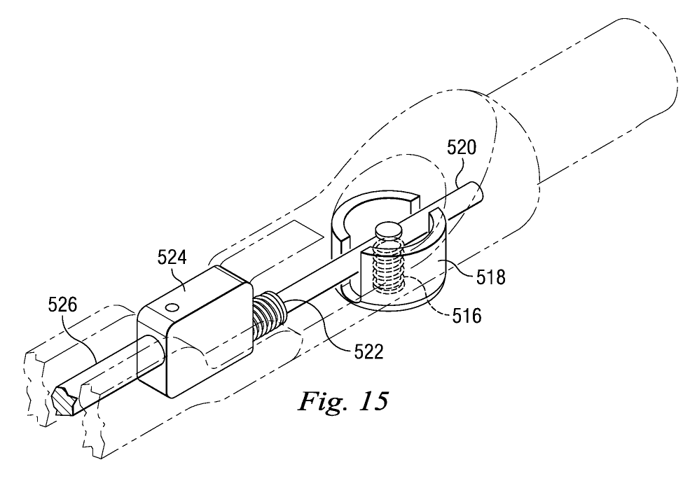

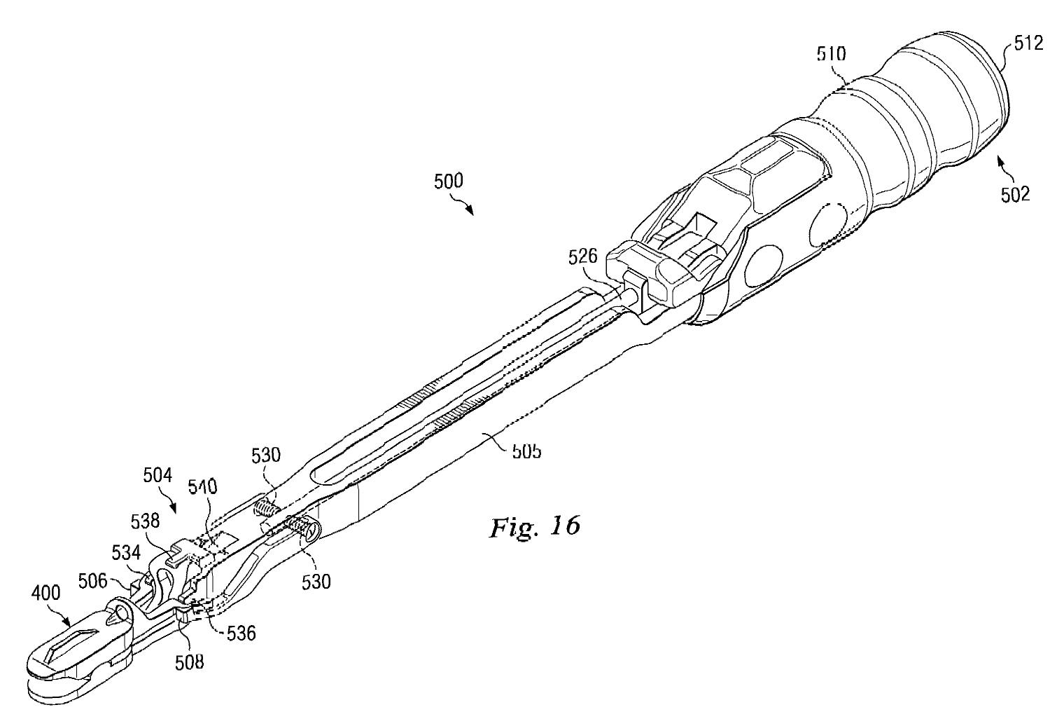

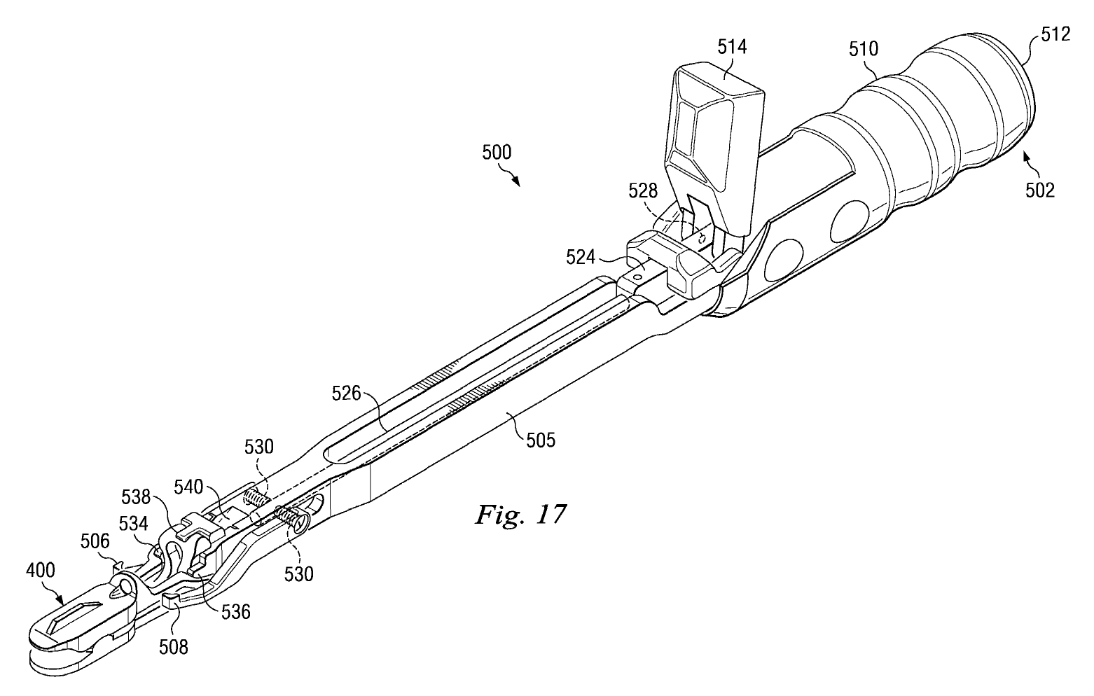

As shown in FIGS. 10 and 11, once the implant 400 is positioned between the vertebrae 7, 9 the guide tube 200 may be positioned into one of the channels of the gripping members 106, 108. In one aspect, a single guide tube 200 is utilized for access to both the superior and inferior portions of the implant and vertebrae—utilizing the same guide tube to access the superior portion first and then the inferior portion, or vice-versa. In another aspect, multiple guide tubes are used. For example, multiple guide tubes may be adapted for simultaneous use to access both superior and inferior portions of the implant and vertebrae. As another example, multiple guide tubes may be utilized where the superior and inferior portions of the implant are secured using different means such that a different sizes or types of guide tubes are appropriate. Where multiple channels are available for orienting the guide tube with respect to the implant, the channel corresponding the implant size or appropriate approach angle is utilized.

Once the guide tube 200 is positioned in the appropriate channel, the drill portion 300 may be inserted through the guide tube 200. The drill portion 300 includes a proximal portion 302 and a bit portion 304. The proximal portion is adapted for engagement with another tool—such as a drill—to facilitate rotation of the bit portion 304. In this way, the bit portion 304 may be utilized to tap or pre-drill the inferior vertebra in preparation to receive a fixation screw 312. Once the inferior vertebra 9 has been tapped the drill portion 300 is removed from the guide tube 200 and the fixation screw 312 is inserted to secure the implant 400 to the inferior vertebra. The fixation screw 312 is inserted by screw driver 310 (see FIG. 1). Next, the guide tube 200 is repositioned in a channel to access the superior vertebra 7. Then the drill portion 300 is again inserted through the guide tube 200 and the superior vertebra 7 is tapped in preparation to receive a fixation screw 314. Again, once the superior vertebra 7 has been tapped the drill portion 300 is removed from the guide tube 200 and the fixation screw 314 is inserted to secure the implant 400 to the superior vertebra. Similar to fixation screw 312, fixation screw 314 is inserted by screw driver 310 (see FIG. 1). FIG. 12 shows the implant 400 inserted and secured to the vertebrae 7, 9 via fixation screws 312, 314, respectively. In some embodiments, the vertebrae 7, 9 are not pre-drilled or tapped prior to insertion of a fixation screw or other mechanism for securing the implant to the vertebrae. In some embodiments, only one of the vertebrae 7, 9 may be tapped.

Referring now to FIGS. 13-17, shown therein is another exemplary embodiment of a surgical instrument 500 according to the present disclosure. Various features of the surgical instrument 500 may be similar to features of the surgical instrument 100 described above. For example, surgical instrument 500 may be used in combination with the guide tube 200, the drill portion 300, the screw driver 310, and the fixation screws 312, 314.

The surgical instrument 500 includes a proximal portion 502, a distal or working portion 504, an elongated body portion 505, and a longitudinal axis L′ extending substantially between the proximal portion and the distal portion. The distal portion 504 comprises two opposed gripping members 506, 508. The gripping members 506, 508 are moveable between a first position for grasping an implant or prosthesis 400 (FIG. 16) and a second position for releasing the implant or prosthesis (FIG. 17). The proximal portion 502 includes a handle 510 and an end portion 512. The handle 510 is adapted for grasping by the user or engagement with another instrument. The end portion 512 is adapted to receive a force from a mallet, hammer, slap-hammer or similar device that is transferred along the length of the surgical instrument and used to urge the implant 400 into position.

Referring now to FIGS. 14 and 15, the surgical instrument 500 also includes a mechanism for moving the gripping members 506, 508 between the positions for grasping and releasing the prosthesis. In the current embodiment, the mechanism includes an actuator 514, a spring 516, a guide body 518, a shaft 520, a spring 522, a portion 524, and an elongated shaft 526. The actuator 514 is positioned adjacent the handle 510 and is adapted for rotational movement about its pivot point 528. The rotational movement of the actuator 514 about pivot point 528 is transferred via the spring 516, the guide body 518, the shaft 520, the spring 522, and the portion 524 into substantially linear motion of the elongated shaft 526 substantially along the longitudinal axis L of the surgical instrument 500. The gripping members 506, 508 are connected to the elongated shaft 526 via screws 530. Thus, the linear motion of the elongated shaft 526 is transferred directly to the gripping members 506, 508. In this manner, the gripping members 506, 508 are moved between the positions for grasping and releasing the prosthesis.

In the current embodiment, the springs 516 and 522 serve to bias the elongated shaft 526 and, therefore, the gripping members 506, 508 toward the proximal portion 502. That is, the springs 516 and 522 serve to bias the gripping members 506, 508 to the grasping position (FIG. 16). Thus, the actuator 514 is utilized to move the gripping members 506, 508 to the release position (FIG. 17). In other embodiments, the gripping members 506, 508 may be biased towards the release position and the actuator 514 utilized to move the gripping members to the grasping position.

In some embodiments, the surgical instrument 500 and the handle 510 are adapted to be held by a single hand of the user and the actuator 514 is adapted for movement by the user's thumb. In one aspect, the actuator 514 includes a locking mechanism (not shown) for selectively securing the actuator in a position. For example, by securing the actuator in a position where the gripping members 506, 508 are engaging the prosthesis the user need not hold the actuator to maintain a secure grip upon the prosthesis. In some embodiments the locking mechanism is combined with biasing the gripping members 506, 508 towards a particular position, either grasping or releasing the implant.

The distal portion 504 of the surgical instrument 500 is adapted to engage the implant 400. To this end, the distal portion 504 includes various features configured to match the shape and contours of the prosthesis 400. For example, in the current embodiment the distal portion 504 includes projections 534, 536, and 538 that mate with corresponding recesses and contours of the implant 400. Similarly, the gripping members 506, 508 are shaped to mate with the implant 400. Thus, when in the grasping position, the gripping members 506, 508, projections 534, 536, 538, and the other features of the distal portion 504 serve to securely grasp the implant 400. It should be noted that gripping member 508 is substantially similar to gripping member 506. In some embodiments, the gripping member 508 is a mirror image of gripping member 506. It should be noted, however, that gripping member 508 is substantially different from gripping member 506 in some embodiments. Further, in some embodiments, the distal portion 504 may include various other features adapted to facilitate secure grasping of the implant 400. The precise features may be configured for the particular implant to be inserted.

The gripping member 106 includes a superior channel 130, a plurality of inferior channels 132, 134, 136, and implant engagement features 138. The implant engagement features 138 are generally shapes, contours, recesses, projections, textures, or other features that facilitate a secure engagement between the surgical instrument 100 and the implant 400. The implant engagement features 138 may be tailored specifically for a particular implant or adapted to accept a variety of implants. In some embodiments, the implant engagement features 138 help prevent any movement of the components of the implant 400 with respect to other components of the implant or the surgical instrument 100 during insertion. For example, where the implant 400 is a motion preserving implant the implant may have multiple components adapted to move with respect to one another. The implant engagement features 138 of the gripping members 106, 108 serve to secure the components of the implant in a fixed orientation during insertion. This facilitates proper alignment of the implant components all the way through insertion and fixation.

The distal portion 504 also includes a superior channel 540. The superior channel 540 is adapted to orient the guide tube 200 such that the guide tube provides access to an inferior portion of the implant 400. In some embodiments, the superior channel 540 positions the guide tube 200 to facilitate fastening of the implant 400 to an inferior vertebra. Similar to the surgical instrument 100, surgical instrument 500 includes inferior channels (not shown) adapted to orient the guide tube 200 such that the guide tube provides access to a superior portion of the implant 400. In some embodiments, one of the inferior channels positions the guide tube 200 to facilitate fastening of the implant 400 to a superior vertebra. As the implant 400 is held securely with respect to the gripping members 506, 508 the superior and inferior channels provide precise alignment of the guide tube 200 with features of the implant. For example, in some embodiments the channels provide precise alignment with an engagement portion of the implant, such as opening adapted to receive fixation screw. In some embodiments, the implant 400 itself serves as a stop to limit the travel of the guide tube 200. In other embodiments, the distal portion 504 of the surgical instrument 500 includes a stop portion to limit the travel of the guide tube 200.

Having a plurality of the inferior channels allows the surgical instrument 500 to be utilized with a variety of implant sizes and types. The plurality of inferior channels may be substantially parallel in some embodiments, and may have different angles providing additional approach angles for the guide tube 200 in other embodiments. In some embodiments, the distal portion 504 includes a plurality of superior channels or no superior channels. Further, in other embodiments the distal portion 504 includes none, one, or more inferior channels. Also, the channels may be replaced by other features such as notches, protrusions, or other mechanisms adapted to engage with a portion of the guide tube 200 to align the guide tube with respect to the implant.

Although only a few exemplary embodiments have been described in detail above, those skilled in the art will readily appreciate that many modifications are possible in the exemplary embodiments without materially departing from the novel teachings and advantages of this disclosure. The current disclosure is applicable to a wide variety of surgical techniques related to the spine, such as total disc arthroplasty, TLIF, and other spine surgeries. Further, while the exemplary embodiments have been described primarily in relation to spinal implants, the current disclosure is applicable to the insertion of all kinds of prostheses.

Accordingly, all such modifications and alternative are intended to be included within the scope of the invention as defined in the following claims. Those skilled in the art should also realize that such modifications and equivalent constructions or methods do not depart from the spirit and scope of the present disclosure, and that they may make various changes, substitutions, and alterations herein without departing from the spirit and scope of the present disclosure. It is understood that all spatial references, such as “horizontal,” “vertical,” “top,” “upper,” “lower,” “bottom,” “left,” and “right,” are for illustrative purposes only and can be varied within the scope of the disclosure. In the claims, means-plus-function clauses are intended to cover the structures described herein as performing the recited function and not only structural equivalents, but also equivalent structures.

What is claimed is:

1. A surgical instrument for inserting an implant between adjacent vertebrae, comprising:
a first grasping member having a first channel for slidably receiving a first portion of a guide tube;
a second grasping member opposed to the first grasping member, the second grasping member having a second channel opposed to the first channel for slidably receiving a second portion of the guide tube; and
an actuator adapted for moving the first and second grasping members between a first position for securely grasping the implant and a second position for releasing the implant.

2. The surgical instrument of claim 1, wherein the first and second channels are adapted to position a distal portion of the guide tube adjacent an engagement portion of the implant.

3. The surgical instrument of claim 2, wherein the engagement portion of the implant is adapted to receive a fixation screw.

4. The surgical instrument of claim 1, wherein the first grasping member further comprises a third channel for slidably receiving the first portion of the guide tube; and
the second grasping member further comprises a fourth channel opposed to the third channel for slidably receiving the second portion of the guide tube.

5. The surgical instrument of claim 4, wherein the first and second channels are adapted to position a distal portion of the guide tube adjacent an inferior engagement portion of the implant; and
the third and fourth channels are adapted to position a distal portion of the guide tube adjacent a superior engagement portion of the implant.

6. The surgical instrument of claim 5, wherein the inferior and superior engagement portions are adapted to receive a fixation screw.

7. The surgical instrument of claim 5, wherein the first grasping member further comprises a fifth channel for slidably receiving the first portion of the guide tube; and
the second grasping member further comprises a sixth channel opposed to the third channel for slidably receiving the second portion of the guide tube.

8. The surgical instrument of claim 7, wherein the fifth and sixth channels are adapted to position a distal portion of the guide tube adjacent a superior engagement portion of the implant.

9. The surgical instrument of claim 8, further including a handle for grasping by a user.

10. The surgical instrument of claim 9, wherein the at least a portion of the actuator is disposed adjacent the handle.

11. The surgical instrument of claim 1, wherein the first and second grasping members are adapted to hold multiple components of the implant in a fixed position with respect to each other when the first and second grasping members are in the first position for securely grasping the implant.

12. The surgical instrument of claim 11, wherein the first and second grasping members are adapted to hold two components of the implant in a fixed position with respect to each other when the first and second grasping members are in the first position for securely grasping the implant.

13. The surgical instrument of claim 11, wherein the first grasping member further comprises a first implant engagement feature adapted to facilitate a secure engagement between the first grasping member and the implant.

14. The surgical instrument of claim 13, wherein the second grasping member further comprises a second implant engagement feature adapted to facilitate a secure engagement between the second grasping member and the implant.

15. The surgical instrument of claim 14, wherein the first and second implant engagement features each comprise a recess.

16. The surgical instrument of claim 14, wherein the first and second implant engagement features each comprise a projection.

17. The surgical instrument of claim 1, wherein the first and second channels are adapted to position at least a portion of the guide tube between the first and second gripping members.

18. A prosthesis inserter, comprising:
an elongated shaft having a proximal portion, a distal portion, and a longitudinal axis extending substantially between the proximal portion and the distal portion;
a handle disposed adjacent the proximal portion;
a gripping portion disposed adjacent the distal portion, the gripping portion adapted to selectively grasp the prosthesis;
a mechanism adapted for moving the gripping portion between a first position for securely grasping the prosthesis and a second position for releasing the prosthesis, the mechanism having a first portion disposed adjacent to the handle; and
a plurality of channels disposed adjacent the distal portion, the plurality of channels adapted to slidably receive at least a portion of a guide tube.

19. The prosthesis inserter of claim 18, wherein at least one of the plurality of channels is adapted to position a distal portion of the guide tube adjacent an inferior portion of the prosthesis.

20. The prosthesis inserter of claim 19, wherein at least one of the plurality of channels is adapted to position a distal portion of the guide tube adjacent a superior portion of the prosthesis.

21. A method of inserting a spinal implant at least partially into the intervertebral space between a first vertebra and a second vertebra, comprising:
providing a surgical instrument comprising a grasping portion movable between a first position for securely grasping the spinal implant and a second position for releasing the spinal implant and a first channel disposed adjacent the grasping portion, the first channel adapted for slidably receiving a portion of a guide tube;
creating a first exposure through a patient's back to access the intervertebral space;
grasping the spinal implant with the surgical instrument;
inserting the spinal implant at least partially into the invertebral space;
sliding the portion of the guide tube into the first channel; and
securing the spinal implant to the first vertebra.

22. The method of claim 21, further comprising pre-drilling the first vertebra prior to securing the spinal implant to the first vertebra.

23. The method of claim 22, wherein securing the spinal implant to the first vertebra comprises inserting a fixation screw through the guide tube and into the first vertebra.

24. The method of claim 21, wherein the surgical instrument further comprises a second channel disposed adjacent the grasping portion adapted to slidably receive a portion of a guide tube.

25. The method of claim 24, further comprising sliding the portion of the guide tube into the second channel and securing the spinal implant to the second vertebra.

26. The method of claim 25, further comprising pre-drilling the second vertebra prior to securing the spinal implant to the second vertebra.

27. The method of claim 26, wherein securing the spinal implant to the second vertebra comprises inserting a fixation screw through the guide tube and into the second vertebra.

28. The method of claim 21, further comprising resecting a portion of the first vertebra.

29. The method of claim 28, wherein resecting a portion of the first vertebra includes creating a recess to receive a keel of the spinal implant.

30. The method of claim 29, further comprising resecting a portion of the second vertebra.

31. The method of claim 30, wherein resecting a portion of the second vertebra includes creating a recess to receive a keel of the spinal implant.
